(19) United States
(12) Patent Application Publication
(10) Pub. No.: US 2015/0301003 A1
(43) Pub. Date: Oct. 22, 2015

(54) METHOD FOR MEASURING THE TRIBOCHARGING PROPERTIES OF BULK GRANULAR MATERIALS AND POWDERS

(71) Applicant: Gregory Peter Martiska, Newtown, CT (US)

(72) Inventor: Gregory Peter Martiska, Newtown, CT (US)

(21) Appl. No.: 14/256,890

(22) Filed: Apr. 18, 2014

(51) Int. Cl. G01N 33/00

(52) U.S. Cl. CPC G01N 33/00; G01N 2033/0091

(57) ABSTRACT

A method is provided for measuring the tribocharging properties of bulk granular materials and powders. A test sample of bulk material is put in contact with a surface of contact material moving in a circular path relative to the test sample under controlled speed and time and the amount of charge generated on the surface of the test sample is measured. Optionally, the friction between the contact surface and the test sample can also be measured. Using this method, many variables that relate to the tribocharging of granular materials can be studied including contact speed between the contact surface and material, contact time between the contact surface and material, pressure between the contact surface and material, and friction between the contact surface and material.

### CROSS-REFERENCE TO RELATED APPLICATIONS

Not Applicable

### BACKGROUND OF THE INVENTION

1) Field of the Invention

Tribocharging is a contact electrification process that enables charge transfer between dissimilar materials due to touching or rubbing of surfaces. This invention relates to a method for measuring the tribocharging properties of bulk granular materials and powders and how these properties change with contacting materials, contacting speeds, contacting times, contacting pressures, and contacting friction. The information produced by these measurements can determine how a material performs in various processes and equipment such as pharmaceutical tableting machines, pneumatics conveyors, container filling machines, and drying systems.

2) Description of the Related Art

Many methods and apparatuses have been disclosed and produced that measure the tribocharging properties of bulk granular materials. The most common method is to allow a known amount of material to slide down a feeding chute, pipe or conduit into a Faraday pail where the total charge on the material is measured. The difference between the charge on the material before and after sliding down the chute or pipe represents the amount of charge accumulated on the material due to frictional contact with the chute. This type of method and apparatus is disclosed in U.S. Pat. No. 6,686,743 and are disclosed in “Triboelectrification of particulate flows on surfaces: Part I-Experiments” by Peter M. Ireland in Powder Technology Volume 198 2010 pages 189-198 and in “Mechanisms of electrostatic charge reduction of granular media with additives on different surfaces” by Saurabh Sarkar, Janet Cho and Bodhisattwa Chaudhuri in Chemical Engineering and Processing 62 2012 pages 168-175.

These methods have many disadvantages. The contact time and speed between the material and contacting chute are difficult to control and change. The contacting pressure cannot be controlled and is only based on the density of the material being tested. Only the total charge on the sample material can be measured and not just the area of the powder that came in contact with the chute material. It is difficult to control the initial charge on the material as the material must be fed slowly into the chute or pipe by another feeding mechanism.

Another method and apparatus for testing static accumulating properties of textile fibers is disclosed in U.S. Pat. No. 2,421,430. In the disclosed system, a sample of textile fibers are put in contact with an electrically isolated rotating plate and the charge produced on the plate is measured using an electrical potential meter connected by wire to the plate. This approach has many disadvantages The contact plate must be electrically isolated for the measurement to have any meaning. This is not typical in manufacturing environments where all material contacting surfaces are grounded. Grounding allows an unlimited source of ions for charging powders unlike an isolated plate. The charge on the surface of the powder cannot be measured directly with this apparatus. Again this does not work if the contact plate is grounded or charged. Also, the measurement scheme of an electrical potential meter in contact with the rotating plate is very ineffective for measuring charge on non-conducting plates. By definition, non-conductors do not conduct charge well so connecting a wire to them will not produce accurate charge measurements.

Many other methods have been disclosed for measuring the tribocharging properties of individual particles. In the method disclosed in “Chargeability measurements of selected pharmaceutical dry powders to assess their electrostatic charge control capabilities” by Juan-Carlos Ramirez-Dorronsoro, Robert B. Jacko,and Dane O. Kildsig in AAPS PharmSciTech 2006 Volume 7 individual particles are carried through a charging pipe by and air stream and the charge developed on them is measured as the particles flow through a ring sensor. In the method disclosed in “A novel sensing technique for measurement of magnitude and polarity of electrostatic charge distribution across individual particles” by Tariq Hussain, Waseem Kaialy, Tong Deng, Mike S. A. Bradley, Ali Nokhodchib, and David Armour-Chelua in International Journal of Pharmaceutics 441 2013 781-789 particles are dropped individually through a charge sensor. In the apparatus disclosed in “Contact charging between nonconductors and metal” by John W. Peterson in Journal of Applied Physics Volume 25 Number 7 July 1954 a single particle is rolled in a cylinder and the charge due to contact between the cylinder and particle is measured. This technique is also used in an apparatus disclosed in “Contact electrification between insulators: phenomenological aspects” by F. R Ruckdeschel and L. P. Hunter in Journal of Applied Physics Volume 46 1975. These methods provide information about individual particle contacts but the data may or may not be relevant to materials sliding together in a powder mass against a tribocharging surface. In addition, the apparatuses disclosed by Peterson and Ruckdeschel are measuring only contact charging between materials as there is no motion between the test particle and rolling cylinder since both particle and cylinder are rolling at the same speed.

Additional methods and apparatus are disclosed in “Tribolelectric charging of powders: a review” by S. Matsusaka, H. Maryuma, T. Matsuyama, and M. Gadhuri in Chemical Engineering Science 65 (2010) 5781-5807. None of the disclosed methods or apparatus allow the charge on the surface of a connected group of particles in the tested sample material to be measured after charging, the friction between the contacting surface and the test material to be measured, or allow the force between the powder mass and contacting surface to be adjusted.

### BRIEF SUMMARY OF THE INVENTION

After working on powder flow testers for many years, the inventors of the present invention determined that industry and academia needed a simple, easy to understand, and inexpensive method to measure the tribocharging properties of bulk granular materials and how these properties change with contacting materials, contacting speeds, contacting pressures, and contacting friction.

The invention consists of a method for measuring the tribocharging properties of bulk granular materials and powders. A test sample of bulk material with a plurality of particles is put in contact with a contact surface of material moving on a circular path relative to the test sample and the amount of charge generated on the surface of the test sample is measured.

Using this method, many variables that relate to the tribocharging of granular materials and powders can be studied. By changing the relative speed between the contact surface and the test sample, the effects of contact speed on tribocharging can be studied. By changing the time that the contact surface is in contact with the contact surface, the effects of contact time on tribocharging can be studied. By changing the pressure that the contact surface exerts on the test sample, the effects of contact pressure on tribocharging can be studied. By measuring the friction between the contact surface and test samples, the effects of friction on tribocharging can be studied. By changing the material that the contact surface is made of, the effects of material on on tribocharging can be studied. These measurements are currently not available to scientists and engineers studying granular materials.

### BRIEF DESCRIPTION OF THE DRAWINGS

FIG. 1 presents a drawing of the preferred embodiment of an apparatus using the invented method with a test sample container with circular inner walls.

FIG. 2 presents a drawing of the preferred embodiment of an apparatus using the invented method with a sample container with annular inner walls.

FIG. 3 presents a drawing of an alternate embodiment of an apparatus using the invented method where the sample container is put in a horizontal position and rotated it along its length axis.

### DETAILED DESCRIPTION OF THE INVENTION

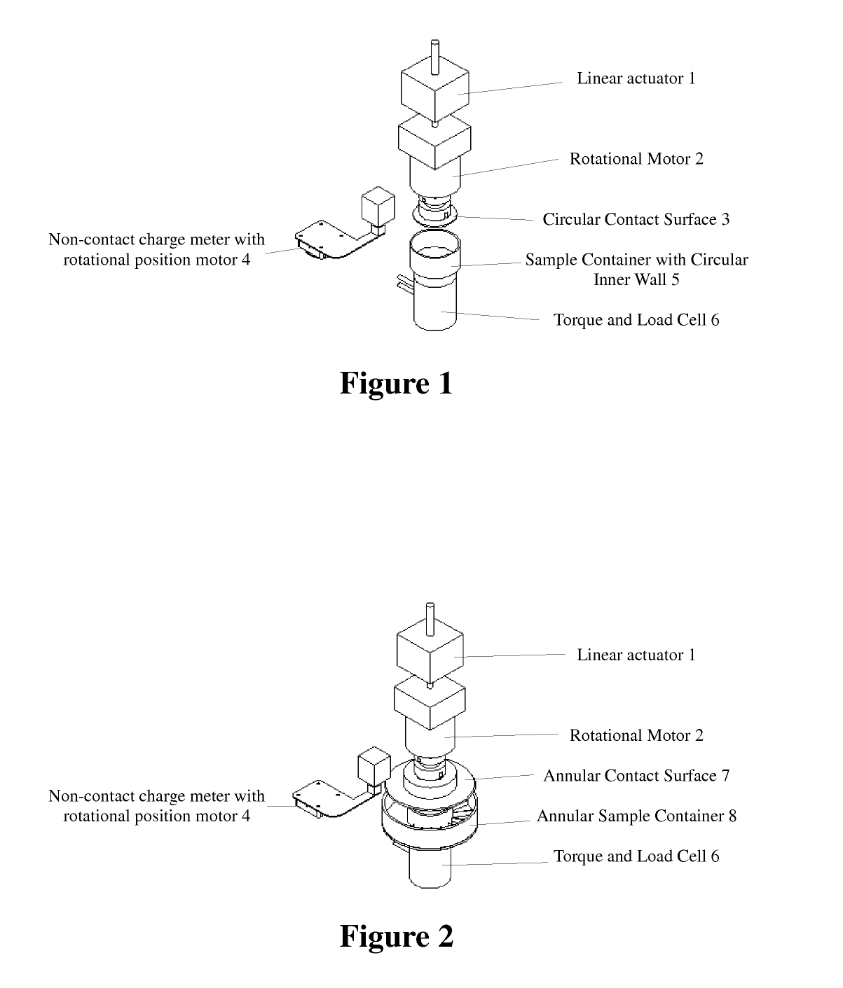

The invention consists of a method for measuring the tribocharging properties of bulk granular materials and powders. The basic idea of the method is to use circular motion to tribocharge a test sample of material and then measure the resulting charge using a non-contacting charge meter. Two preferred embodiments of apparatus to employ the invented method are presented in FIGS. 1 and 2. The method consists of the following steps:
1) A test sample of bulk granular material or powder with a plurality of particles is put in a sample container with a circular inner wall and optionally including another inner wall to create an annular container. The preferred sample container with a circular inner wall 5 is illustrated in FIG. 1 and the preferred sample container with inner wall to create an annular container is illustrated in FIG. 2 as annular sample container 8.
2) A contact surface is positioned so it is in contact with the test sample. The preferred contact surface for the circular sample container consists of a flat circular surface of material illustrated as circular contact surface 3 in FIG. 1. The preferred surface for the annular sample container consists of a flat circular surface of material with a hole in the center to create an annular surface illustrated as annular contact surface 7 in FIG. 2.
3) A known pressure can be created and maintained between the test sample and contact surface. This is possible if the contact surface has dimensions smaller that the sample container and covers most of the surface of the test sample. The preferred apparatus for creating and maintaining a known pressure between the test sample and contact surface consists of a force sensor and a linear actuator. The force sensor is illustrated as torque and load cell 6 in FIGS. 1 and 2. The linear actuator is illustrated as linear actuator 1 in FIGS. 1 and 2.
4) Frictional motion on a circular path between the test sample and contact surface is then created at a known speed for a known time. The preferred apparatus rotates the contact surface using a rotational motor illustrated as rotational motor 2 in FIGS. 1 and 2 while the sample container is stationary. The frictional force between the contact surface and test sample can optionally be measured during this rotation. The preferred apparatus for measuring the frictional force between the contact surface and test sample is a reaction torque sensor as illustrated as torque and load cell 6 in FIGS. 1 and 2.
5) The electrical charge created by the frictional motion between the test sample and contact surface is measured using a non-contacting charge meter. The preferred apparatus for the non-contacting charge meter is a chopper stabilized field meter illustrated as non-contact charge meter with rotational position motor 4 in FIGS. 1 and 2. The preferred technique is to remove the contact surface and position the charge meter above the surface of the test sample. Optionally, the charge on the contact surface can be measured.

Using this method, various aspects of the tribocharginng properties of bulk granular materials and powders can be studied. By varying the pressure on the contact surface, the effects of pressure and friction on the tribocharging of the test material can be studied. By varying the speed of the circular motion between the contact surface and the test sample, the effects of velocity on the tribocharging of the test material can be studied. By varying the contact surface material and smoothness, the effects of material and surface smoothness on the tribocharging of the test material can be studied.

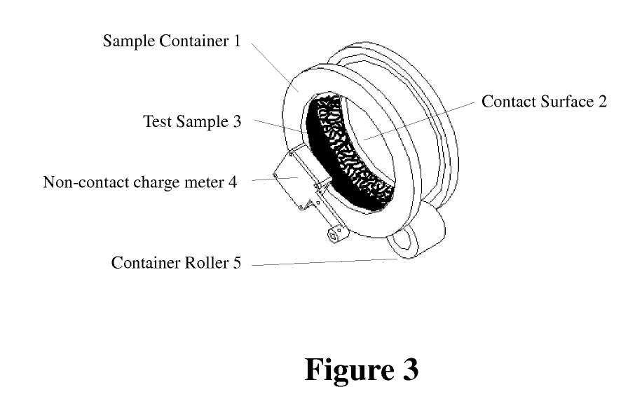

An alternative apparatus to the two preferred embodiments of apparatus to employ the invented method is presented in FIG. 3. In this apparatus, the frictional motion along a circular path between the test sample and contact surface is created by putting a sample container is a horizontal position and rotating it along its length axis. The test sample will remain essentially in one position while the contact surface moves against its surface.

In FIG. 3, sample container 1 is rotated by container roller 5. This causes frictional motion along a circular path between test sample 3 and contact surface 2. The electrical charge created by the frictional motion between the test sample and contact surface is measured using a chopper stabilized field meter labeled as non-contact charge meter 4 that is positioned in front of contact surface 2.

The alternative apparatus is not the preferred embodiment of an apparatus to employ the invented method because it does allow the pressure between the test sample and contact surface to be changed nor does it allow the friction between the test sample and contact surface to be measured during the frictional motion between the test sample and contact surface. However it is much simpler than the preferred embodiment so it is less expensive to produce. In addition, it has the advantage that the test sample can be isolated from environmental conditions. This is important when controlling humidity during the test and for samples that may be toxic.

What is claimed is:

1. A method for measuring the tribocharging properties of bulk granular material and powders comprising a) loading a test sample of granular material or powder with a plurality of particles into a sample container with a circular inner wall; b) positioning a contact surface so it is in contact with the test sample; c) creating motion on a circular path between the test sample and contact surface at a known speed and for a known time; d) using a non-contacting charge measuring device to measure the electrical charge created by the motion between the test sample and contact surface.

2. A method for measuring the tribocharging properties of bulk granular material and powders according to claim 1 where an additional inner circular wall is added to the sample container with a circular inner wall and a corresponding circular hole is added to the contacting surface to create an annular contact surface between the test sample and the contacting surface.

3. A method for measuring the tribocharging properties of bulk granular material and powders according to claims 1 and 2 where the motion on a circular path between the test sample and contact surface is created by rotating the contact surface and holding the sample container in a fixed position.

4. A method for measuring the tribocharging properties of bulk granular material and powders according to claims 1 and 2 where the motion on a circular path between the test sample and contact surface is created by rotating the sample container and holding the contact surface in a fixed position.

5. A method for measuring the tribocharging properties of bulk granular material and powders according to claim 1 where the motion on a circular path between the test sample and contact surface is created by placing the sample container in a horizontal position and rotating it along its length axis

6. A method for measuring the tribocharging properties of bulk granular material and powders according to claim 1 or claim 2 where the friction between the test sample and contact surface is measured during the frictional motion in step c.

7. A method for measuring the tribocharging properties of bulk granular material and powders according to claim 1, claim 2 or claim 5 where the electrical charge created by the motion between the test sample and contact surface is measured with the contact surface in contact with the test sample.

8. A method for measuring the tribocharging properties of bulk granular material and powders according to claim 1, claim 2, or claim 5 where the electrical charge created by the motion between the test sample and contact surface is measured after the contact surface is no longer in contact with the test sample.
